(19) United States
(12) Patent Application Publication
(10) Pub. No.: US 2024/0335283 A1
(43) Pub. Date: Oct. 10, 2024

(54) MOTORIZED IMPLANT DELIVERY DEVICE, IMPLANT, LOADING SYSTEM, AND METHOD OF USING

(71) Applicant: EDWARDS LIFESCIENCES CORPORATION, Irvine, CA (US)

(72) Inventors: Robert Foster Wilson, Roseville, MN (US); Kris Siverhus, Maple Grove, MN (US); Steve Zaver, Plymouth, MN (US); Zach Garvey, Stillwater, MN (US); Tom Gerold, Monticello, MN (US); David Elizondo, Pondview, MN (US); Sean Miller, Maple Grove, MN (US)

(21) Appl. No.: 18/744,559

(22) Filed: Jun. 14, 2024

Related U.S. Application Data
(63) Continuation of application No. PCT/US2022/080936, filed on Dec. 5, 2022.
(60) Provisional application No. 63/369,011, filed on Jul. 21, 2022.
(60) Provisional application No. 63/265,586, filed on Dec. 17, 2021.

(51) Int. Cl. A61F 2/24

(52) U.S. Cl. CPC A61F 2/243; A61F 2/2418; A61F 2/2436; A61F 2220/0075

(57) ABSTRACT

A heart valve implantation device with a motor and drive mechanism that allows a single operator to deliver a prosthetic heart valve to a target site. The motor controls relative positions between a delivery catheter and an implant release mechanism attached to the implant. The motor can be operated in two directions, allowing the motor to be used not only for delivery, but for loading and extraction of the valve, if necessary. An embodiment of a valve implant and a loading system designed for use with the device are also described.

### RELATED APPLICATIONS

The present application is a continuation of Patent Cooperation Treaty Application Number PCT/US2022/080936, filed on Dec. 5, 2022, titled “MOTORIZED IMPLANT DELIVERY DEVICE, IMPLANT, LOADING SYSTEM, AND METHOD OF USING”, which claims the benefit of U.S. Provisional Application Ser. No. 63/265,586, filed on Dec. 17, 2021, and claims the benefit of U.S. Provisional Application Ser. No. 63/369,011, filed on Jul. 21, 2022, which are all incorporated by reference in their entireties.

### BACKGROUND

There has been a significant movement toward developing and performing cardiovascular surgeries using a percutaneous approach. Through the use of one or more catheters that are introduced through, for example, the femoral artery, tools, and devices can be delivered to a desired area in the cardiovascular system to perform any number of complicated procedures that normally otherwise require an invasive surgical procedure. Such approaches greatly reduce the trauma endured by the patient and can significantly reduce recovery periods. The percutaneous approach is particularly attractive as an alternative to performing open-heart surgery.

Valve replacement surgery provides one example of an area where percutaneous solutions are being developed. A number of diseases result in a thickening, and subsequent immobility or reduced mobility, of heart valve leaflets. Such immobility also may lead to a narrowing, or stenosis, of the passageway through the valve. The increased resistance to blood flow that a stenosed valve presents can eventually lead to heart failure and ultimately death.

Treating valve stenosis or regurgitation has heretofore involved complete removal of the existing native valve through an open-heart procedure followed by the implantation of a prosthetic valve. Naturally, this is a heavily invasive procedure and inflicts great trauma on the body leading usually to great discomfort and considerable recovery time. It is also a sophisticated procedure that requires great expertise and talent to perform.

Historically, such valve replacement surgery has been performed using traditional open-heart surgery where the chest is opened, the heart stopped, the patient placed on cardiopulmonary bypass, the native valve excised, and the replacement valve attached. A proposed percutaneous valve replacement alternative method on the other hand, is disclosed in U.S. Pat. No. 6,168,614 (the entire contents of which are hereby incorporated by reference) issued to Andersen et al. In this patent, the prosthetic valve is mounted on a stent that is collapsed to a size that fits within a catheter. The catheter is then inserted into the patient's vasculature and moved so as to position the collapsed stent at the location of the native valve. A deployment mechanism is activated that expands the stent containing the replacement valve against the valve cusps. The expanded structure includes a stent configured to have a valve shape with valve leaflet supports begins to take on the function of the native valve. As a result, a full valve replacement has been achieved but at a significantly reduced physical impact to the patient.

However, this approach has decided shortcomings. One particular drawback with the percutaneous approach disclosed in the Andersen '614 patent is the difficulty in preventing leakage around the perimeter of the new valve after implantation. Since the tissue of the native valve remains within the lumen, there is a strong likelihood that the commissural junctions and fusion points of the valve tissue (as pushed apart and fixed by the stent) will make sealing around the prosthetic valve difficult. In practice, this has often led to severe leakage of blood around the stent apparatus.

Other drawbacks of the Andersen '614 approach pertain to its reliance on stents as support scaffolding for the prosthetic valve. First, stents can create emboli when they expand. Second, stents are typically not effective at trapping the emboli they dislodge, cither during or after deployment. Third, stents do not typically conform to the features of the native lumen in which they are placed, making a prosthetic valve housed within a stent subject to paravalvular leakage. Fourth, stents are subject to a tradeoff between strength and compressibility. Fifth, stents cannot be retrieved once deployed. Sixth, stents have an inherent strength that is not adjustable.

As to the first drawback, stents usually fall into one of two categories: self expanding stents and balloon expandable stents. Self-expanding stents are compressed when loaded into a catheter and expand to their original, non-com pressed size when released from the catheter. These are typically made of Nitinol. Balloon expandable stents are loaded into a catheter in a compressed but relaxed state. These are typically made from stainless steel or other malleable metals. A balloon is placed within the stent. Upon deployment, the catheter is retracted, and the balloon inflated, thereby expanding the stent to a desired size. Both of these stent types exhibit significant force upon expansion. The force is usually strong enough to crack or deform thrombosis, thereby causing pieces of atherosclerotic plaque to dislodge and become emboli. If the stent is being implanted to treat a stenosed vessel, a certain degree of such expansion is desirable. However, if the stent is merely being implanted to displace native valves, less force may be desirable to reduce the chance of creating emboli. An additional concern related to displacing an aortic valve is the risk of conduction disturbances (i.e., left bundle branch block) due to the close proximity of the conduction pathways to the native valve structure. Excessive radial force applied at the native valve site increases the risk of irritation or damage to the conduction pathway and heart block.

As to the second drawback, if emboli are created, expanded stents usually have members that are too spaced apart to be effective to trap any dislodged material. Often, secondary precautions must be taken including the use of nets and irrigation ports.

The third drawback results from the relative inflexibility of stents. Stents typically rely on the elastic nature of the native vessel to conform around the stent. Stents used to open a restricted vessel do not require a seal between the vessel and the stent. However, when using a stent to displace native valves and house a prosthetic valve, a seal between the stent and the vessel is necessary to prevent paravalvular leakage. Due to the nonconforming nature of stents, this seal is hard to achieve, especially when displacing stenosed valve leaflets.

The fourth drawback is the tradeoff between compressibility and strength. Stents are made stronger or larger by manufacturing them with thicker members. Stronger stents are thus not as compressible as weaker stents. Most stents suitable for use in a valve are not compressible enough to be placed in a thin catheter, such as an 18 Fr catheter. Larger delivery catheters are more difficult to maneuver to a target area and also result in more trauma to the patient.

The fifth drawback of stents is that they are not easily retrievable. Once deployed, a stent may not be recompressed and drawn back into the catheter for repositioning due to the non-elastic deformation (stainless steel) or the radial force required to maintain the stent in place (Nitinol). Thus, if a physician is unsatisfied with the deployed location or orientation of a stent, there is little he or she can do to correct the problem.

The sixth drawback listed above is that stents have an inherent strength and are thus not adjustable. As previously stated, stronger stents are made with stronger members. Once a stent is selected and deployed, there is little a physician can do if the stent proves to be too strong or too weak.

Various embodiments of devices that solve these problems are in a family of patents to Thill et al., entitled “Stentless Support Structure,” that includes U.S. Pat. Nos. 8,974,523; 9,271,831; 9,180,002; 9,439,761; 9,168,132; 9,439,760; as well as numerous pending and foreign applications and patents, the contents of which are incorporated herein in their entireties. These patents teach a braided mesh tube that is capable of folding back and forth into itself to build, in situ, a support structure that is strong enough to hold back the leaflets of a native valve sufficiently to successfully deploy a replacement valve, thus obviating the need for excision of the native valve. Advantageously, because of the inverting nature of these devices, the braided mesh, in an elongated delivery configuration, does not need to possess the strength to accomplish native valve displacement until the inversion process occurs. This allows the mesh tube to be constructed such that, in the elongated delivery state, the tube can be compressed into a very small catheter, such as an 18 Fr or smaller catheter. Such a small catheter significantly reduces patient trauma and allows for easy percutaneous, intraluminal navigation through the blood vessels. It is to be understood that terms like transluminal and percutaneous, as used herein, are expressly defined as navigation to a target location through and axially along the lumen of a blood vessel or blood vessels as opposed to surgically cutting the target vessel or heart open and installing the device manually. It is further to be understood that the term “mesh” as used herein describes a material constructed of one or more braided or woven strands.

In order to accomplish the folding back and forth feature of this device, there are preformed, circumferential folds in the device. One embodiment has two circumferential folds that are longitudinally spaced apart in the extended configuration. One of these folds is preformed to fold inwardly, and the other is preformed to fold outwardly. These preformed folds, when released out of a catheter, tend to return to a folded configuration that has a z-like cross-section. This cross-section design results not only because the inward pre-formed fold folds inwardly and the outward pre-formed fold folds outwardly, but because these folds reverse longitudinal positions once folded. If the inward preformed fold is distal of the outward preformed fold in the extended position, in the folded position the inward preformed fold will be proximal of the outward preformed fold. This design allows a valve on a distal end of the device to be drawn into the device when folded, without requiring the valve itself to be inverted or everted. In one embodiment having two preformed folds, the inversion process thus results in a three-layered configuration that could be significantly shorter than the extended length, depending on the spacing of the folds.

A delivery device was developed specifically for the delivery of such an implant. An early iteration of this delivery device is shown and described at least in U.S. Pat. No. 9,795,478, to Wilson et al., entitled Inversion Delivery Device and Method for a Prosthesis, the contents of which are incorporated by reference herein. This delivery device included a plurality of control cables that allowed a physician to control the rate at which the implant was expelled from the distal end of the device, and also allowed the proper operation of the implanted valve prior to completely releasing the implant. If the physician was not satisfied with the positioning of the implant, the cables could be used to pull the valve back into the delivery catheter and relocate the valve to a desired site.

The aforementioned device, however, included several different knobs and buttons and took time to learn proper usage as a result. Furthermore, the physician relied on tactile feedback combined with visual fluoroscopic feedback to determine the timing of the various steps in the procedure.

To address these concerns, a next generation of the delivery device was developed. This delivery device is shown and described in at least U.S. Pat. No. 10,820,995 to Czyscon et al. entitled Inversion Delivery Device and Method for a Prosthesis. This delivery device was designed to flatten the learning curve for using it by providing a positioning mechanism that automatically initiates the inversion process once a predetermined length of the implant has exited the delivery catheter. The device used a combination of a carriage and follower arms that interact with a leadscrew to change the direction of the carriage travel while maintaining a manual rotation of a knob in a single direction to rotate the leadscrew.

This device greatly increased the case of use during a delivery but still required a two-hand operation, at a minimum, and often required two people to operate. There is a need for a device that can be safely and effectively operated by a single person.

In addition to the need for an improved delivery system, mechanical heart valves present numerous design challenges that must be overcome in order to achieve efficacy. Just a few of these challenges include being able to be delivered easily, accurately and atraumatically; being able to be loaded into a delivery device without damaging the device; being able to withstand hundreds of millions of cycles without suffering performance degradations; and being able to be implanted securely such that valve migration or paravalvular leakage does not occur. The list of design considerations is long and a mechanical heart valve may never be created that functions as well as a healthy native valve. As such, there is always a need for an improved prosthetic valve.

One difficulty that designing a prosthetic heart valve presents is attaching or anchoring the implant to a target attachment site. The leaflets of a native valve are pliable and grow directly out of the conduit through which fluid is being regulated. Prosthetic valves, especially those being delivered from a catheter, typically include a pliable leaflet material attached to a rigid supporting structure such as a stent or a wireform or a combination thereof. Delivering such a device can place stresses on the soft leaflet material as it gets ejected from the delivery catheter. Thus, there is a need for an implant design that protects the delicate leaflets during the delivery process.

Paravalvular leakage is another concern that needs addressing. Paravalvular leakage refers to blood that makes its around a prosthetic valve implant instead of through the leaflets. This leakage results in regurgitant flow and reduced valvular efficacy. Optimally atraumatic implantation techniques involve pushing the native valve leaflets out of the way as opposed to excising the leaflets. This often results in an irregular implantation site geometry. The support structure must be pliable enough to conform to the implantation site geometry, thus creating a seal between the implant and the target site, while still providing enough support to form the coapting valve leaflets and anchor the implant securely. The design must also prevent leakage between the leaflets and the internal surfaces of the support structure.

### SUMMARY

The present application is directed toward a device that addresses the need for an automated delivery device that allows one-handed operation. This need is addressed by providing a motorized delivery device that completes all or most of the delivery steps by pressing a button that activates a motor. Additionally, a heart valve is described herein that was developed to work with the delivery device that is constructed to protect the soft leaflet material during delivery by preventing contact between the leaflet material and the delivery catheter. The implant also includes a skirt and a liner that, when the implant is in a folded configuration, prevents paravalvular leakage.

One aspect of the invention is a cardiac valve implant that includes a tubular braided support structure having an unfolded configuration and a folded configuration. The tubular support structure has a proximal end formed of a plurality of spires. Each spire has a high point, and low points on either side of the high points. The support structure further includes a distal end, and first and second circumferential preformed folds between the distal and proximal ends of the tubular braided support structure. The circumferential preformed folds bias the tubular braided support structure toward the folded configuration.

In at least one embodiment of the invention the cardiac valve implant also includes a valve leaflet assembly having a wireform with a plurality of commissural points separated by arcuate portions and valve material attached to the wireform. The wireform shapes the valve material into coapting valve leaflets when the valve material is attached to the wireform.

In at least one embodiment, the valve leaflet assembly is attached to an inside surface of the support structure such that the commissural points of the wireform are aligned with the spires of the support structure. This ensures the soft valve material does not come into contact with the delivery catheter during loading or delivery as the braided support structure acts as a barrier between the catheter and the valve assembly.

One aspect of the invention provides a cardiac valve implant that includes a tubular braided support structure having an unfolded configuration and a folded configuration. The support structure has a distal end, a proximal end, and first and second circumferential preformed folds between the distal and proximal ends of the tubular braided support structure. The circumferential preformed folds bias the tubular braided support structure toward the folded configuration.

In at least one embodiment the implant also includes a valve leaflet assembly having a wireform including a plurality of commissural points separated by arcuate portions and valve material attached to the wireform. The wireform shapes the valve material into coapting valve leaflets when the valve material is attached to the wireform the valve leaflet assembly is attached to an inside surface of the support structure such that the valve material is protected from contact with a delivery catheter by the support structure.

Another aspect of the invention is a support structure for a cardiac valve implant that has a proximal end formed of a plurality of spires each having a high point and low points on either side of the high points 326; a distal end; and first and second circumferential preformed folds between the distal and proximal ends of the support structure, the circumferential preformed folds biasing the support structure toward a folded configuration.

In at least one embodiment, when the support structure is in the folded configuration, the support structure forms a three-layered middle region, a single-layered proximal region, and a single-layered distal region. The single-layered proximal region comprises proximal portions of the plurality of spires, which include the highpoints.

In at least one embodiment, the invention provides A delivery device for an implant comprising: a control cable having a distal end attachable to an implant and a proximal end; a delivery catheter surrounding the control cable and having a distal end and a proximal end; and, a handle assembly adjustably connected to the proximal ends of the control cable and the delivery catheter and including: a motor; a battery pack capable of powering the motor; at least one control connected between the battery pack and the motor, usable to provide power having a first polarity that powers the motor in a first direction and usable to provide power having a second polarity that powers the motor in a second direction; a drive mechanism that moves the control cable in a distal direction and the delivery catheter in a proximal direction relative to the handle when the motor is powered in the first direction, and moves the control cable in a proximal direction and the delivery catheter in a distal direction when the motor is powered in the second direction.

Another aspect of the invention is a delivery system for delivering an implant to a target site within a patient comprising: a motorized delivery device having a handle, a delivery catheter assembly extending distally from the handle, and a motor contained within the handle and operable to pull a connector into a delivery catheter during a loading procedure and retract the delivery catheter relative to the implant during a delivery procedure; a loading tray usable to assist in loading the implant into the delivery device and including: a handle compartment sized and shaped to hold the handle in a desired position; a loading basin usable to contain a solution during a loading procedure; and, a channel leading from the handle compartment to the loading basin and sized to retain the delivery catheter during a loading procedure.

Yet another aspect of the invention is a method of delivering an implant to a target location comprising: loading an implant into a distal end of a delivery catheter; navigating the distal end of the delivery catheter to a target location; activating a motor in a first direction within a handle associated to a proximal end of the delivery catheter that retracts relative to the implant, thereby allowing the implant to expand within the target location; and, releasing the implant.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a schematic diagram of the basic components of an embodiment of the invention;

FIG. 2 is a plan view of an embodiment of a delivery catheter assembly of the invention;

FIG. 3 is a plan view of an embodiment of a nose cone according to the invention;

FIG. 4 is a plan view of an embodiment of a leadscrew according to the invention;

FIG. 5 is a side elevation of an embodiment of a handle assembly, with a cover removed to show internal components, according to the invention;

FIG. 6 is a perspective view of an embodiment of a handle assembly according to the invention;

FIG. 7 is a perspective view of an embodiment of a valve connector according to the invention;

FIG. 8 is a perspective view of an embodiment of an implant according to the invention;

FIG. 9 is a front elevation of an embodiment of a support structure according to the invention in an unfolded configuration;

FIG. 10 is a perspective view of an embodiment of the support structure according to the invention;

FIG. 11 is a perspective view of an embodiment of the implant of the invention in a relaxed state;

FIG. 12 is a cross-sectional profile view of an embodiment of a folded support structure of the invention;

FIG. 13 is a perspective view of an embodiment of a valve assembly according to the invention showing the wireform and valve material;

FIG. 14 is an elevation of an embodiment of a wireform according to the invention;

FIG. 15 is a distal end view of an embodiment of the implant according to the invention in a non-compressed, non-folded configuration;

FIG. 16 is a perspective view of a delivery device packaged in an embodiment of an accessory kit of the invention;

FIG. 17 is a side elevation of an embodiment of a loading tool according to the invention;

FIG. 18; is a sectional view of the loading tool of FIG. 17 taken along section lines A-A; and,

FIG. 19 is a side elevation of an embodiment of a loading tool adapter according to the invention.

### DETAILED DESCRIPTION

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

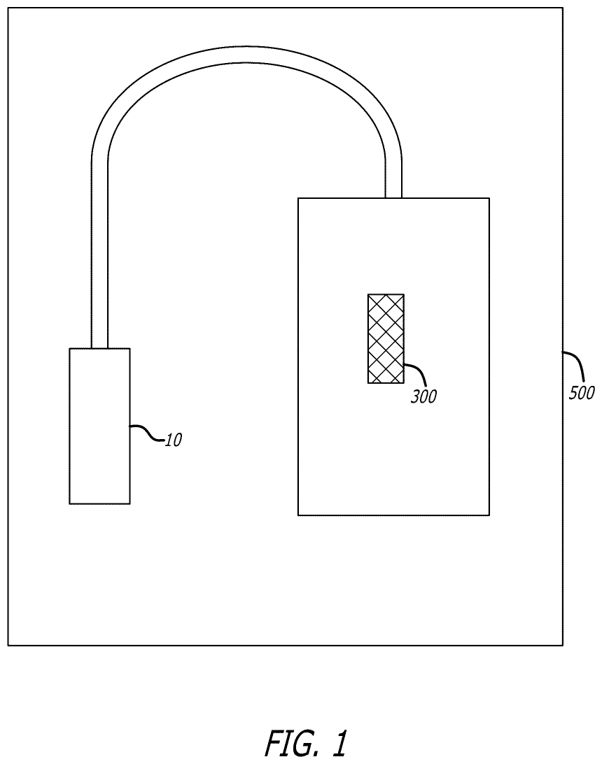

Referring first to FIG. 1 there is a schematic diagram of the basic components of the invention. The invention generally includes a delivery device 10, an implant 300 and an accessory kit 500.

### Delivery Device

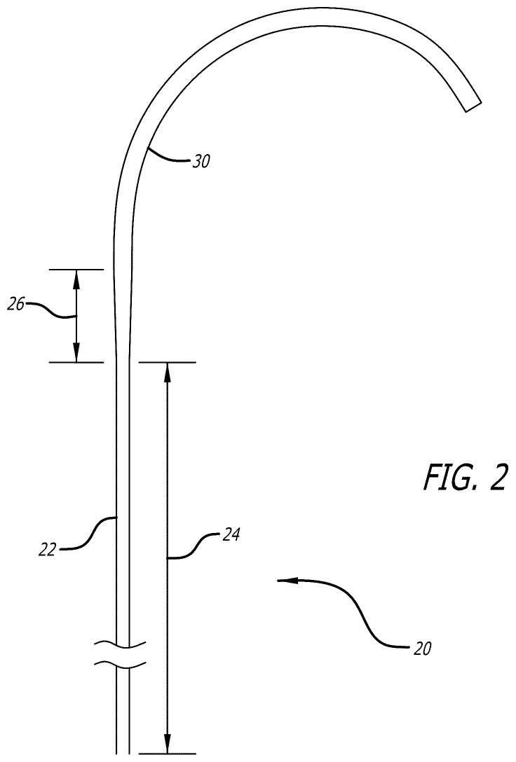

The delivery device 10 generally includes a delivery catheter assembly 20, a nose cone 50, and handle assembly 100. FIG. 2 shows the delivery catheter assembly 20, which in at least one embodiment includes a shaped delivery catheter 22. The delivery catheter 22 is reinforced with axial fibers along its length that provide improved strength, durability, and deliverability. Additionally, the delivery catheter 22 has a hydrophilic coating that improves trackability.

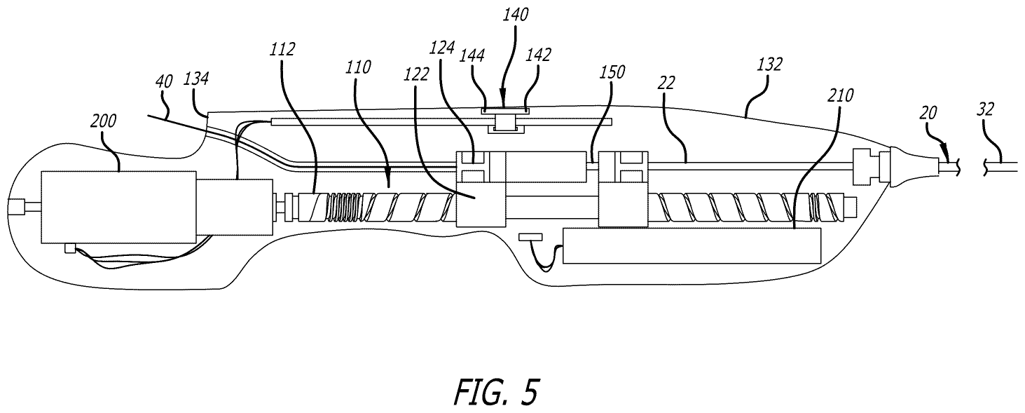

The delivery catheter 22 has a straight section 24 that extends from the handle assembly 100 (see FIG. 5) from its proximal end to a capsule 30 at its distal end. In at least one embodiment, the straight section 24 has an outer diameter of between 0.150 inches and 0.250 inches. Optimal results have been achieved with an outer diameter of approximately 0.185 inches. The straight section 24 has an inner diameter of between 0.100 inches and 0.200 inches. Optimal results have been achieved with an inner diameter of approximately 0.155 inches. As will be seen, the capsule 30 carries the valve to the desired location, and thus has slightly larger inner and outer diameters. As such, the straight section 24 transitions to the capsule 30 with a taper 26 at its distal end, approximately 1.0 inch in length.

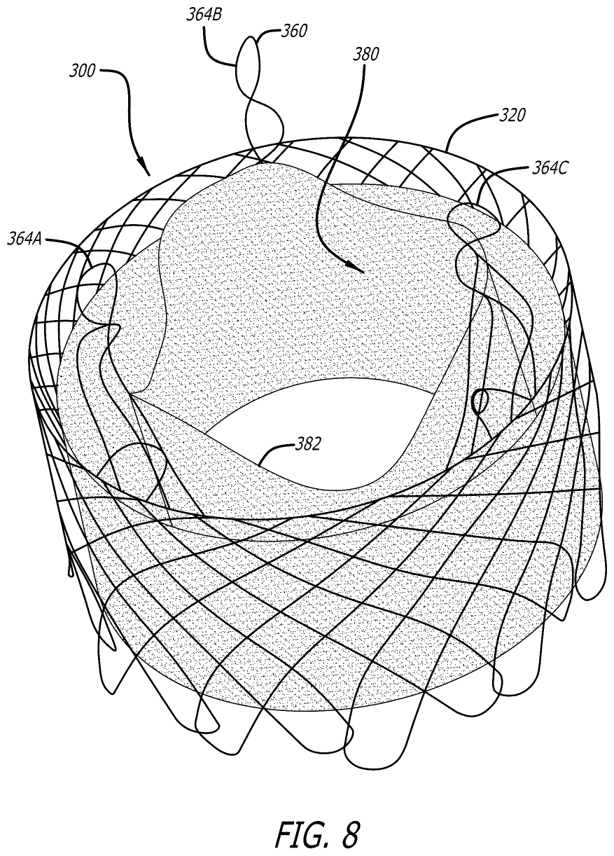

The capsule 30 is a pre-shaped curved section of catheter 22 that extends distally from the straight section 24 and carries the valve implant 300 (see FIG. 8). The curved capsule 30, in at least one embodiment, has an average radius of between 1.5 inches and 2.5 inches. Optimal results have been achieved with an average radius of approximately 1.92 inches. In at least one embodiment the curve has a decreasing radius from proximal end to distal end. For example, in one embodiment, the capsule 30 transitions from the straight section 24 with a radius of approximately 2.3 inches at approximately 45 degrees from the straight section 24 to a radius of approximately 1.85 inches approximately 90 degrees from the straight section 24 to a radius of approximately 1.4 degrees approximately 135 degrees from the straight section 24.

In at least one embodiment, the curved capsule 30 has an outer diameter of between 0.200 inches and 0.300 inches, with optimal results being achieved with an outer diameter of approximately 0.236 inches. In at least one embodiment, the curved capsule 30 has an inner diameter of between 0.150 and 0.250 inches, with optimal results shown at approximately 0.208 inches. The delivery catheter 22 slides within an outer sheath 32, which is connected to the handle assembly 100.

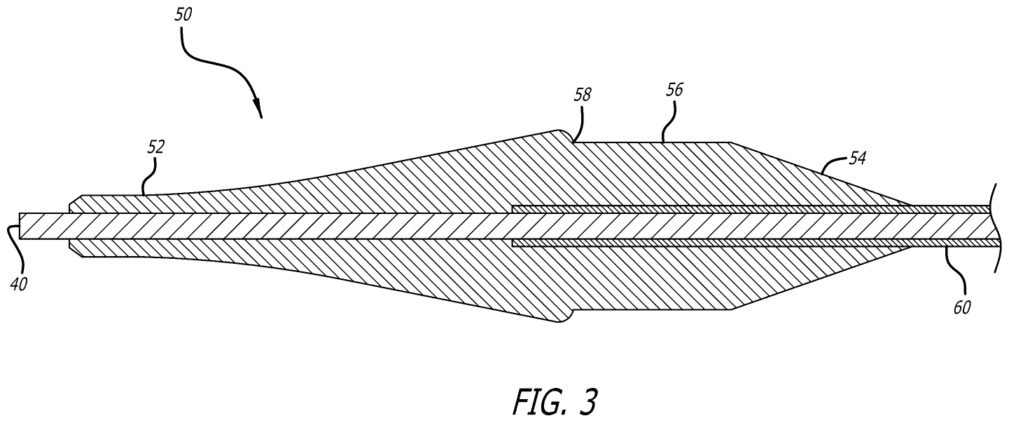

The nose cone 50 is best shown in FIG. 3. The nose cone 50 is located at the distal end of the curved capsule 30 and is shaped to be inserted and easily removed from the distal end of the capsule 30. The shaped catheter 22 is designed to be passed over a guidewire 40 that has been navigated to the target site. The nose cone 50 provides an atraumatic delivery of the catheter to the target location while passing over the guidewire 40.

The nose cone 50 is preferably made of a soft material. In at least one embodiment the nose cone 50 has a durometer of less than 40 D. Optimal results have been achieved with a material having a durometer of 35 D. The nose cone 50 has a distal leading taper 52, a proximal taper 54, and a cylindrical portion 56 between the leading and proximal tapers, 52 and 54. The proximal taper 54 assists in recapturing the nose cone 50 after implantation has occurred. Additionally, the proximal taper 54 cases nose cone insertion into the distal end of the catheter 22 during assembly.

The cylindrical portion 56 is sized to maintain proper alignment of the distal end of the catheter 22 while still being able to be advanced out of the end of the catheter 22 when the implant is delivered. For example, optimal results have been demonstrated with a cylindrical portion 56 having an outer diameter of between 0.200 inches to 0.206 inches when used with a catheter having an inner diameter of 0.208 inches at its distal end.

The leading taper 52 is sized to be flush with the outer diameter of the catheter, thereby creating a smooth transition between the catheter and the nose cone 50. The difference in diameter between the cylindrical portion 56 and the proximal end of the leading taper 52 creates a shoulder 58 that is approximately the same height as the distal end of the catheter 22. This results in the aforementioned smooth transition and provides a stop when inserting the nose cone 50 into the distal end of the catheter 22. In one embodiment, the leading taper 52 has a diameter of between 0.232 and 0.238 inches and tapers to a distal end having a diameter of approximately 0.040 inches.

Like the catheter 22, the nose cone 50 includes a hydrophilic coating to improve lubricity for tracking. Additionally, nose cone 50 has a guidewire lumen 60 running through length of the nose cone 50 and the distal end of the guidewire lumen 60, where the valve contacts the proximal taper 54, also has a hydrophilic coating to reduce sliding friction between the valve and the guidewire lumen 60. In at least one embodiment, the nose cone 50 and the guidewire 40 are decoupled from the valve actuation to provide nose cone depth stability during implant deployment.

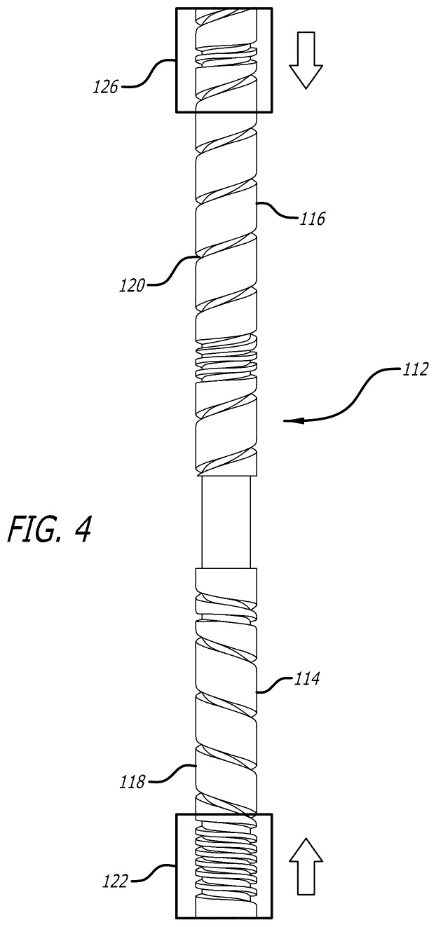

The handle assembly 100 is shown in FIGS. 4-6 and generally includes a drive assembly 110, a motor assembly 200 that powers the drive assembly 110, a housing 132, and a plurality of controls 140 for controlling the motor assembly 200 and releasing the implant 300 during implantation. The motor assembly 200 is powered by a battery pack 210.

The drive assembly 110 includes a leadscrew 112 that creates relative motions between the implant 300 and the catheter 22 when rotated. Referring to FIG. 4, it can be seen that the leadscrew 112 includes a proximal portion 114 and a distal portion 116. The proximal portion 114 includes left-handed threads 118 of varying pitch. A valve nut 122 is engaged with the threads and translates axially when the leadscrew 112 is rotated. The valve nut 122 acts against an adjacent valve carriage 124 that is connected to a valve retention cable 150. The valve retention cable 150 is connected to the implant 300 via three valve connectors 152 that extend from the distal end of the valve retention cable 150, which is trifurcated.

The distal end of the leadscrew 112 includes right-handed threads 120 of varying pitch. A catheter nut 126 is engaged with the right-handed threads 120 and translates axially when the leadscrew 112 is rotated. The catheter nut 126 acts against an adjacent catheter carriage 128 (see FIG. 5) that is connected to the delivery catheter 22, which translates axially within the outer sheath 32. The outer sheath 32 is fixed to the distal end of the handle housing 132.

As further seen in FIG. 5, the valve carriage 124 and the catheter carriage 128 are aligned axially with the catheter assembly 20. The distal end of the valve carriage 124 is connected to the proximal end of the valve retention cable 150. The valve retention cable 150 passes through the catheter carriage 128 and enters the proximal end of a lumen of the delivery catheter 22.

The varying pitch of the left-handed and right-handed threads 118 and 120 controls the speeds and directions at which the carriages 124 and 128 translate. These speeds are determined by the optimal speeds of the delivery catheter 22 in relationship to the implant 300 during implantation and retraction. These speeds will be explained in further detail in the operation discussion below. The differing lengths of the left-handed and right-handed threads 118 and 120 accommodate for the varying pitches such that the same number of rotations of the leadscrew 112 are required for both carriages 124 and 128 to reach their respective travel limits.

The controls 140 of the handle assembly 100 include at least an advance button 142 and a retract button 144. The advance button 142 is a switch that connects power from the battery pack 210 to the motor assembly 200 and results in rotation of the leadscrew 112 in a first direction. The retract button 144 is located proximally of the advance button 142 and is a switch that connects power from the battery pack 210 to the motor assembly 200 in a polarity opposite that of the advance button thereby causing the motor assembly 200 to rotate in a second direction, and rotating the leadscrew 112 in the second direction. One skilled in the art will realize that numerous switch and button configurations could be used in order to achieve this result without departing from the spirit of the invention.

The handle assembly 100 includes a guidewire port 134 near a proximal end of the housing 132. The guidewire port 134 allows the delivery device 10 to be passed over a guidewire 40. When doing so, the guidewire 40 runs through the cone assembly, and through a guidewire lumen 60 formed within the delivery catheter 22, and out the guidewire port 134.

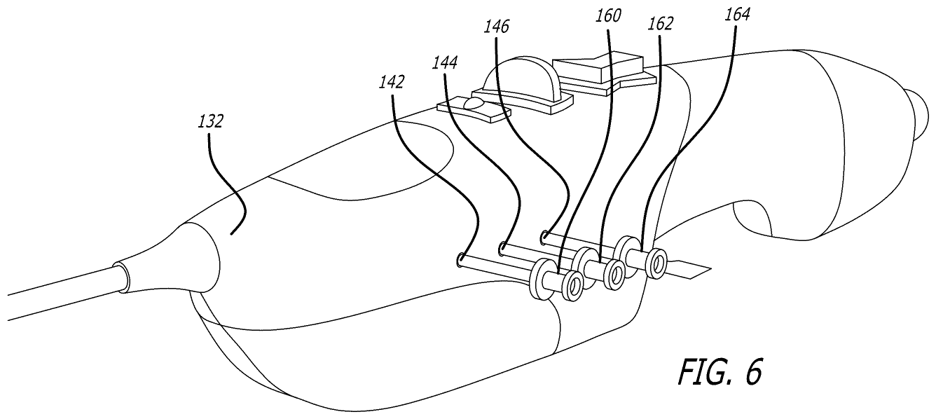

As can be seen in FIG. 6, the handle assembly 100 also includes three flush ports 146, 147 and 148 on a side of the handle housing 132. The flush ports 146, 147, 148 are used to flush air out of the luminal spaces within the delivery system prior to use in a patient. The flush ports 146, 147, 148 include luer connectors 160, 162, and 164 to connect a flushing syringe to in order to force air out of the delivery system's luminal space after loading the implant.

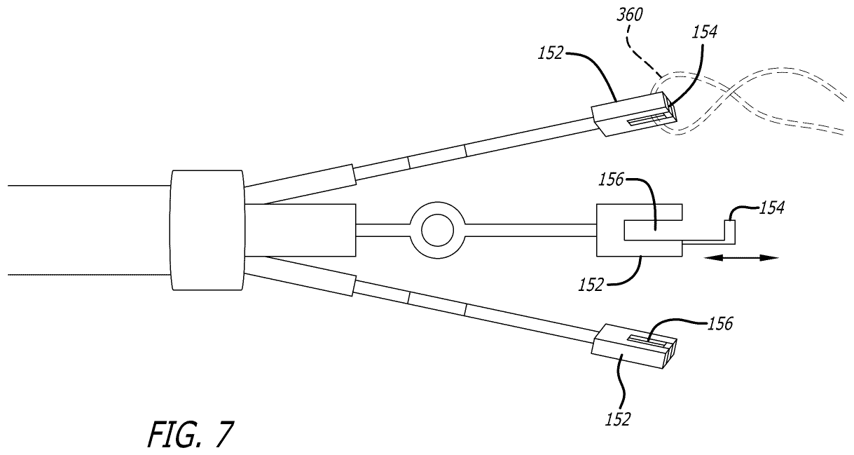

FIG. 7 illustrates that the valve connectors 152 each have a tooth 154 that can extend from a mouth 156 to release the implant 300.

### Implant

Referring now to FIG. 8, the implant 300 generally includes a support structure 320 that folds upon being released from the catheter 22, and a valve assembly 380 that includes valve leaflet material 382 and a wireform 360 to which the valve leaflet material 382 is attached. The valve leaflet material 382 and the wireform are configured such that, when the valve leaflet material 382 and the wireform are attached to each other, a valve assembly 380 is formed that includes valve leaflets that mimic the function of a healthy human heart valve.

The support structure 320 folds longitudinally and expands radially when released from the delivery catheter 22 such that it pushes native valve tissue outwardly, anchors itself in place, holds and orients the wireform and leaflet assembly in an optimal position, and prevents blood from leaking around the prosthetic valve in a retrograde direction, referred to herein as “paravalvular leakage.”

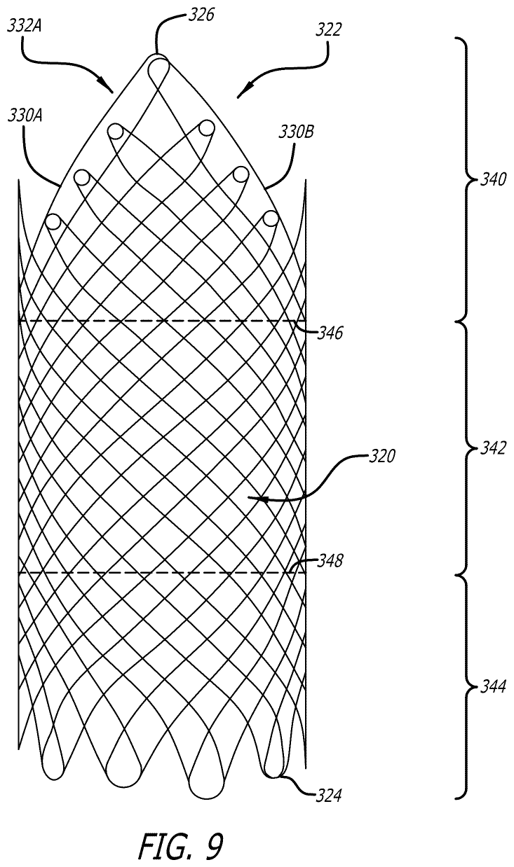

Referring to FIG. 9, there is shown a front elevation of an embodiment of a support structure 320 of the invention in an unfolded configuration. For purposes of clearly showing the braid pattern, the support structure 320 in FIG. 9 has not yet been heat-set and is thus cylindrical. It is also noted that the support structure 320 of FIG. 9 is shown as though it is placed on a solid dowel or mandrel so that the back half of the structure is hidden from view. This provides a clearer view of the braid pattern of the support structure 320.

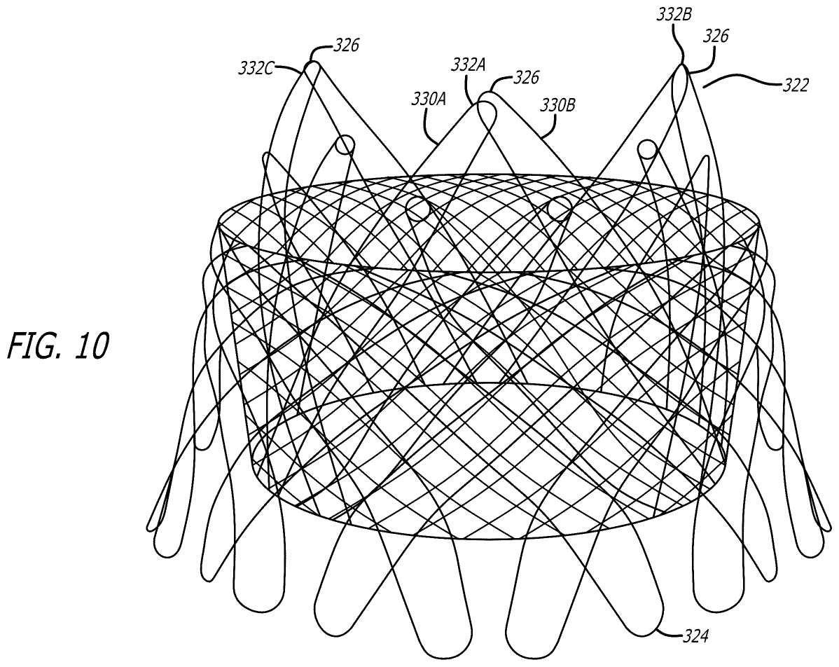

The support structure 320 is a braided structure that has a first end 322 and a second end 324. The first end 322 includes three high points 326, only one of which can be seen in FIG. 9 because the back portion of the support structure 320 is not shown. FIG. 10 shows all three high points 326. Each of the high points 326 are formed by two long braids 330A and 330B, that extend in opposite directions and terminate where they intersect with each other to form the high point 326. All of the remaining braids are of varying length such that they terminate (change directions) at a point that is roughly aligned with the braids 330A and 330B that form the high points 326. The result is a trifurcated first end 322 that has three spires 332A, 332B and 332C (see FIGS. 9 and 10), the function of which will be explained below.

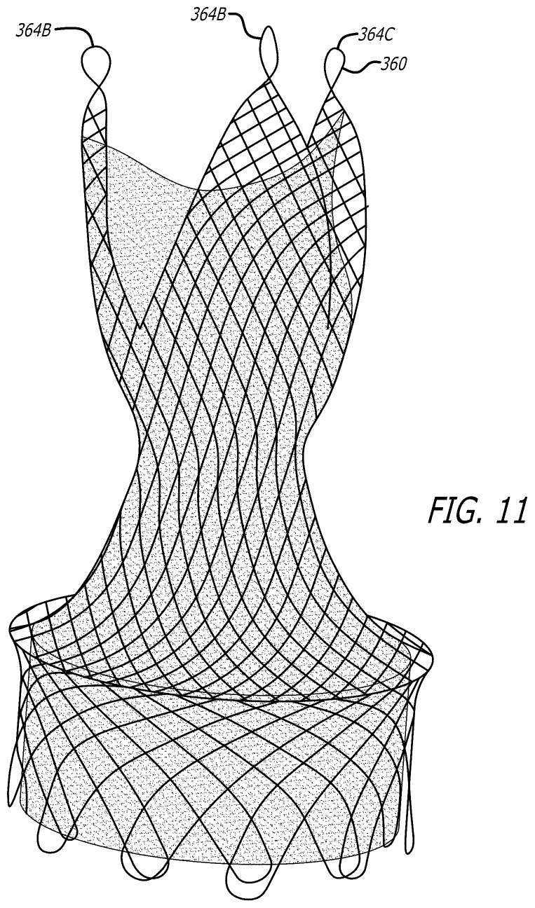

The support structure 320 is divided into three longitudinal sections 340, 342 and 344. The longitudinal sections are separated by two preformed folds 346 and 348, which are shown as dotted lines in FIG. 9 and can be seen in an unfolded, relaxed state in FIG. 11. The preformed folds 346 and 348, at a minimum, assist in reconfiguring the support structure 320 from an unfolded configuration during deployment, to a folded configuration when released from the delivery device 10.

The first longitudinal section 340 includes the first end 322 of the support structure 320, and extends to below where the spires 332A, 332B and 332C intersect with adjacent spires. The second longitudinal section 342 is located between the first fold 346 and the second fold 348. The third longitudinal section 344 extends from the second fold 348 to the second end 324 of the support structure 320.

The second end 324 of the support structure 320 terminates in a plurality of braids that have a different braiding pattern than that of the second longitudinal section 342. The pitch of the braids results in ventricular flair loops 328 that assist in anchoring the support structure 320 to the native valve. These loops, as shown in FIG. 10, flare outwardly to assist in anchoring the implant 300.

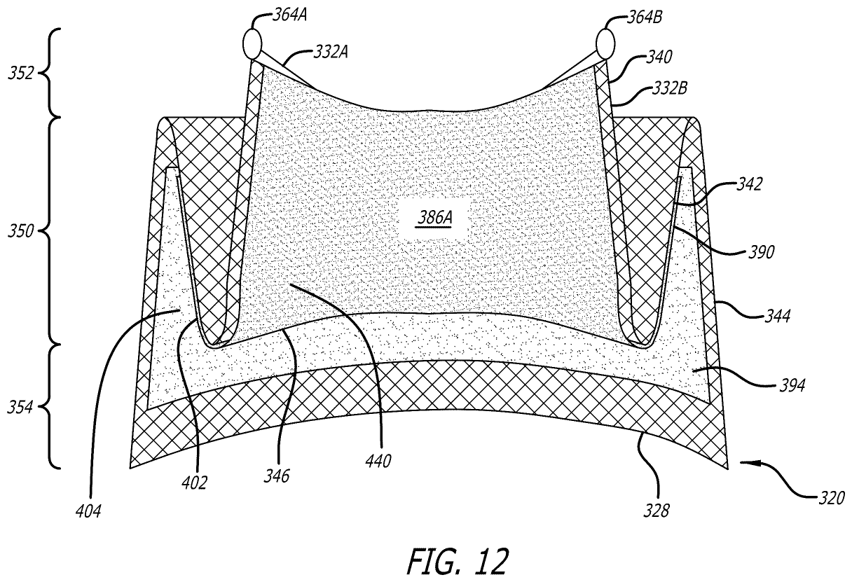

FIG. 10 shows the support structure 320 in a folded configuration. For purposes of descriptive clarity, and because during a typical non-transapical delivery, the second end 324 exits the delivery device first, the second end 324 will be designated as the distal end and the first end 322 will be designated as the proximal end. In the unfolded state, the first preformed fold 346 is located proximal of the second preformed fold 348. In the folded configuration, the second longitudinal section 342 inverts, such that an outside face of the second longitudinal section faces inward. Outside faces of the first and third longitudinal sections do not invert and remain facing outward. When the second longitudinal section 342 inverts, the first section 340 is drawn into the second section, and the first and second sections are pulled into the third section 344. Thus, as shown in FIG. 12, the three longitudinal sections become nested within each other, and are no longer longitudinally adjacent. Rather, section 340 becomes the inner layer, section 342 becomes the middle layer, and section 344 becomes the outer layer. FIG. 12 is a simplistic cross-sectional view showing the three sections 340, 342 and 344 in a folded configuration.

Further shown in FIGS. 10 and 12, the three segments of support structure 320, when folded, result in a device having a three-layered middle region 350, a single-layered proximal region 352, and a single-layered distal region 354. The proximal region 352 is formed by the tips of the spires 332A, 332B and 332C. The distal region 354 is formed by the ends of the ventricular flair loops 328.

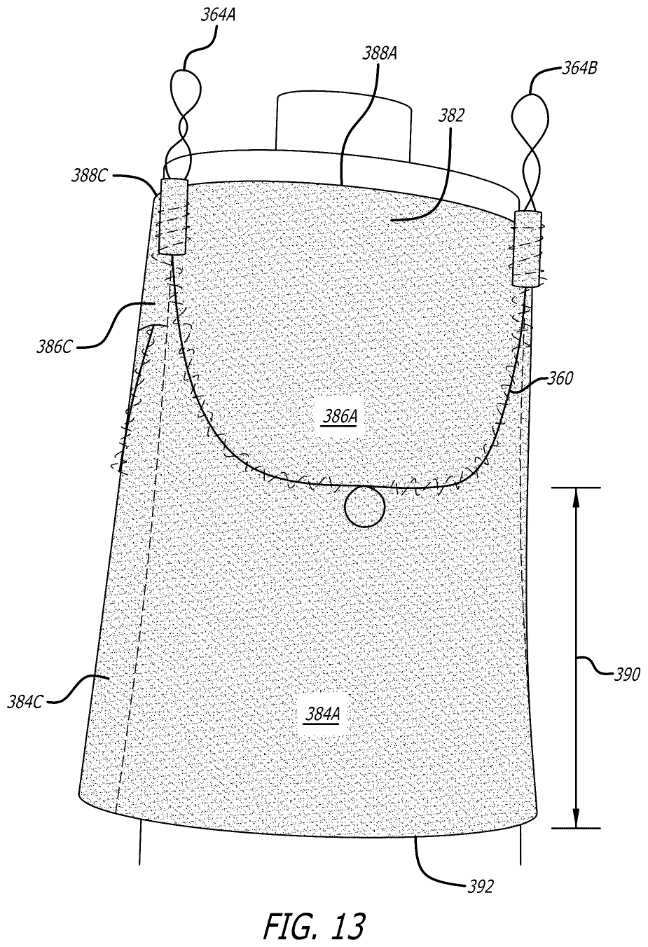

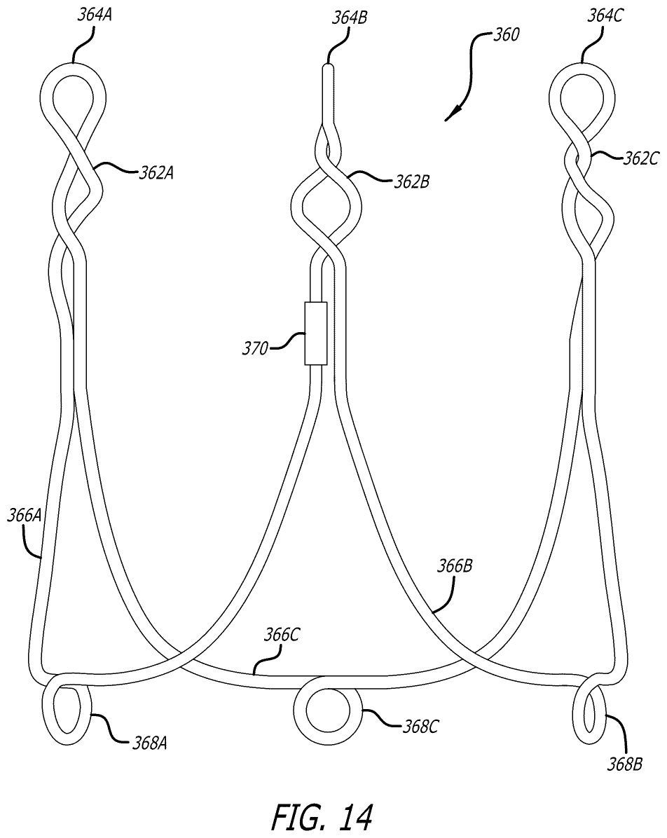

As stated above, the implant 300 includes a valve assembly 380 (FIG. 13) that includes a wireform 360 and tissue 382, such as porcine tissue. FIG. 14 shows an embodiment of the wireform 360 of the invention. The wireform 360 includes three commissural points 362A, 362B and 362C. The commissural points have at their tips, loops 364A, 364B and 364C that are used as attachment points to connect the prosthetic valve implant 300 to the valve connectors 152 of the delivery device 10. Each of the loops 364A, 364B and 364C are angled differently in order to operate optimally with connections of the delivery device 10. Each of the loops 364A, 364B and 364C are angled such that a vertical plane that roughly contains the loop intersects with the similar planes of the two other loops near an axial center of the wireform 360. The commissural points 362A, 362B and 362C are separated by arcuate portions 366A, 366B and 366C. At the midpoint of each arcuate portion 366A, 366B, 366C is a loop 368A, 368B, and 368C, respectively. In the embodiment of FIG. 14, the wireform 360 is made of a single wire, the two ends of which are connected end-to-end with a connector 370.

Referring again to FIG. 13, the wireform 360 is used to create a valve assembly 380 using valve material 382, which may be harvested tissue, such as porcine or bovine tissue, or it may be a synthetic tissue. In at least one embodiment, the valve material 382 includes three separate sheets of material 384A, 384B and 384C, sewn, welded, or otherwise joined with each other and with the wireform 360 to form three valve leaflets 386A, 386B and 386C. In FIG. 13, sheet 384A and leaflet 386A are shown clearly, sheet 384C and leaflet 386C are on the left side, and sheet 384B and leaflet 386B are obscured from view.

In at least one embodiment, each of the sheets 384A, 384B and 384C are longitudinally longer than the wireform 360. The sheets 384A, 384B and 384C are each attached, using sutures or other attachment techniques or materials, to the wireform 360 such that a proximal edge 388A, 388B and 388C are located just distal of the loops 364A, 364B and 364C. The proximal edges 388A, 388B and 388C form the coapting edges of the valve assembly 380. The location of the loops 364A, 364B and 364C proximal of the edges 388A, 388B and 388C ensures that the loops, and corresponding connectors 152 of the delivery device 10, will not interfere with the opening and closing of the valve leaflets 386A, 386B and 386C during delivery. This allows the implant 300 to function fully even while the implant 300 is still connected to the delivery device 10. Thus, verification of correct placement and function can be made prior to release. If the implant 300 is not placed correctly, the implant 300 can be drawn back into the delivery device 10 and relocated.

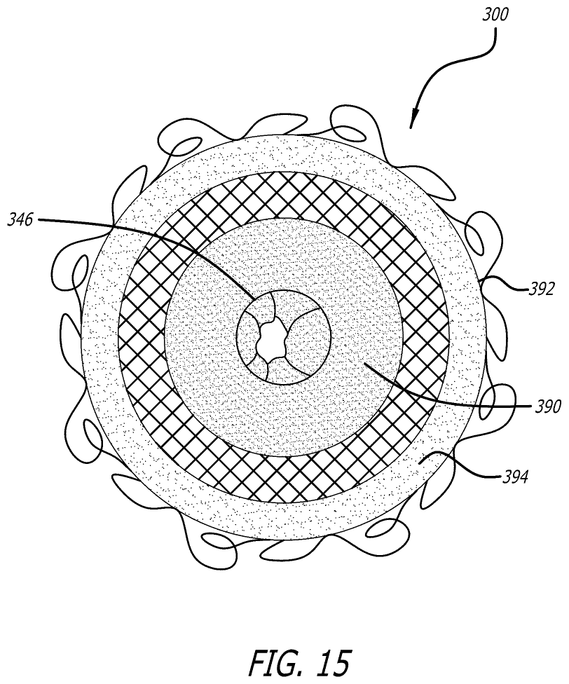

The material of the sheets 384A, 384B and 384C that extends distal of the wireform 360 is sewn or otherwise joined together to form a skirt 390 with a distal edge 392. In at least one embodiment, the distal edge 392 is located distally of the more proximal preformed fold 346. Thus, as seen in FIGS. 12 and 15, when the support structure 320 folds, the skirt 390 folds, forming an internal seal within the support structure 320 that prevents paravalvular leakage.

FIG. 15 is a distal end view of a non-compressed, non-folded configuration of the implant 300. An inside view of the distal edge 392 of the skirt 390 is shown, and the proximal preformed fold 346 can also be seen. Referring back to FIG. 11, which is a perspective view of the outside of a non-compressed, non-folded configuration of the implant 300, the placement of the skirt 390, the wireform 360, and the proximal and distal preformed folds 346 and 348, respectively, can be seen. FIG. 11 also shows how the valve assembly 380 is completely protected from contact with the inside surfaces of the delivery device 10 by the support structure 320.

Additionally, there is an optional distal liner 394 that extends around an internal surface of the distal end of the support structure 320. Referring back to FIG. 12, the distal liner 394 acts in conjunction with the skirt 390 to form an “articulated skirt”. In the folded configuration, the tissue layers can be described as having an internal layer 400 made up of the leaflets 386A, 386B, and 386C and the part of the skirt 390 that extends distally to the first preformed fold 346. The tissue skirt 390 follows around the fold 346 and continues proximally to create a middle layer 402. The liner 394 can be described as an outer-most layer 404 that is located on the inside surface of the distal end of the support structure 320 and faces the middle layer 402. When the support structure 320 is folded and compressed by the implantation site, the middle layer 402 and the outer-most layer 404 are compressed together to form a seal that prevents paravalvular leakage. The gap between the proximal edge of the liner 394 and the distal edge 392 of the skirt 390 ensures that no bunching or other adverse effects occur when the support structure 320 folds.

### Accessory Kit

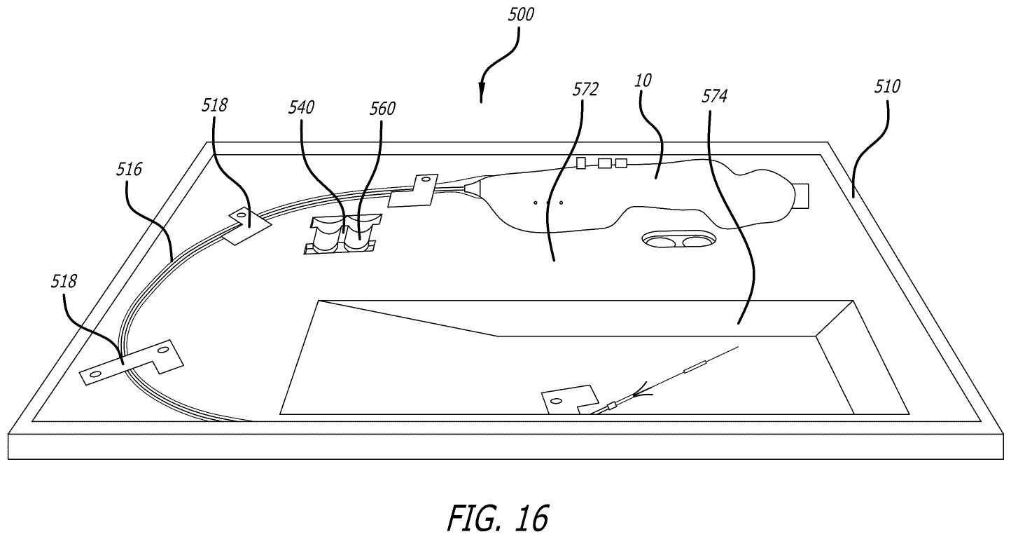

Referring now to FIG. 16, the accessory kit 500 may include a loading tray 510, a loading tool 540, and a loading tool adapter 560. The loading tray 510, combined with a lid (not shown), also serves as the packaging for the system. The loading tray 510 is rigid and has been developed to allow for a single operator to load a implant 300 into the delivery device 10. The tray 510 includes a handle compartment 512, a loading basin 514, and a channel 516 that leads from the handle compartment 512 to the loading basin 514. The channel 516 is curved and houses the delivery catheter 22. The channel 516 has retaining clips 518 covering the channel 516 so that the delivery catheter 22 stays within the channel 516 after the lid is removed. The loading basin 514 provides a place where a implant 300 may be rinsed prior to loading.

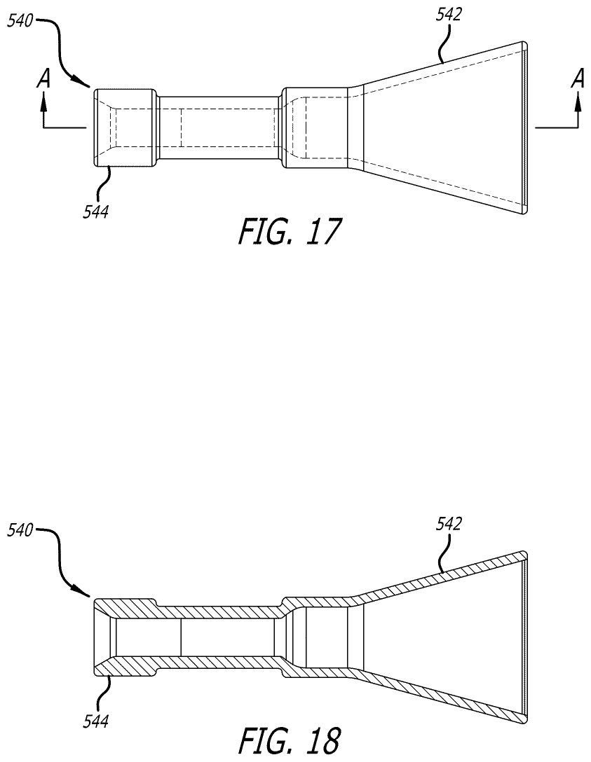

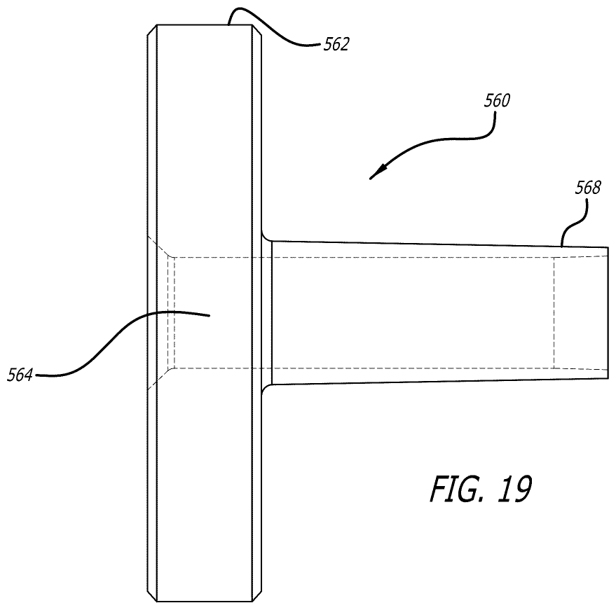

FIGS. 17 and 18 show an embodiment of the loading tool 540, which is a funnel like device that allows the implant 300 to be loaded into the distal end of the delivery catheter 22. The loading tool 540 has a wide side 542 and a narrow side 544. The loading tool 540 works in conjunction with the loading tool adapter 560, shown in FIG. 19, which has a flange 562 with a diameter sized to raise the tip of the catheter 22 to a desired height and support the tip while loading the implant. In at least one embodiment, the loading tool 540 is made from a clear polycarbonate and the loading tool adapter 560 is made from Delrin. The flange 562 has a central lumen 564 sized to accept the distal tip of the delivery catheter 22. The other end 568 has an outer diameter that is sized to fit into the narrow side 544 of the loading tool 540. Operation

Operation begins with implant 300 loading. The lid is removed from the tray 510 and the rinse basin 514 is filled with a solution such as saline. The implant 300 is then placed in the solution and the distal end of the delivery catheter 22 is placed into the flanged end of the valve loading tool adapter 560. The other end of the adapter 560 is inserted into the narrow side 544 of the valve loading tool 540.

Next, the operator pushes the advance button 142, causing the leadscrew 112 to rotate in a direction that translates the catheter 22 carriage and the valve carriage 124 away from each other to the outer extents of the leadscrew 112. The separation of the carriages retracts the catheter 22 proximally while advancing the valve control cable distally, exposing the trifurcated end of the valve control cable for loading the implant 300.

Next the operator opens the valve connectors using the valve connector controls. The loops of the commissural points are each placed in one of the open connectors and the valve connector controls are used to close the connectors.

The implant 300 is now prepared for loading into the delivery device 10. The operator presses the retract button 144 and the motor assembly 200 drives the leadscrew 112, causing the valve carriage 124 and the catheter carriage 128 to translate away from each other, drawing the implant 300 proximally into the distal end of the catheter 22 through the valve loading tool 540 and the valve loading tool adapter 560.

The operator continues to press the retract button 144 until the implant 300 is completely inside the capsule 30 and ready for delivery. The valve loading tool 540 and adapter 560 are then removed from the distal end of the catheter 22 and the delivery device 10 is ready to be removed from the tray 510, after opening the retaining clips 518, when the physician is ready to perform the implant 300.

When ready, physician navigates a guidewire 40 through the damaged native valve. The proximal end of the guidewire 40 is then inserted into the distal end of the nose cone 50 and the nose cone 50 is then inserted into the distal end of the catheter 22, aided by the proximal taper 54 of the nose cone 50. The delivery device 10 is advanced over the guidewire 40 until the distal end of the catheter 22 is at the target site as verified by fluoroscopy, with the aid of radiopaque bands at the distal end of the outer sheath 32. As the delivery device 10 is advanced over the guidewire 40, the proximal end of the guidewire 40 passes through the guidewire port of the handle.

When satisfied with the location of the distal end of the delivery device 10, the physician presses the advance button 142, which turns the leadscrew 112 and causes the valve and catheter carriages 124 and 128 to translate toward each other, and the implant 300 begins to emerge from the distal end of the delivery catheter 22. The third longitudinal section of the implant 300 expands against the damaged valve and begins to expand the native valve.

As the physician continues to hold the advance button 142, the interaction between the third longitudinal section and the native valve assists in holding the implant 300 in place while the second section of the implant 300 is advanced and inverted into the third longitudinal section. The inversion is aided by the second preformed fold reassuming the folded configuration to which it is biased.

It is important to note that at any point in the procedure prior to releasing the implant 300, the physician can release the advance button 142 and, if unhappy with the placement of the implant 300, depress the retract button 144 and draw the implant 300 back into the delivery catheter 22. This also allows the physician to quickly abort the procedure entirely and remove the delivery device 10 from the patient.

Continuing to depress the advance button 142, the delivery catheter 22 continues to move proximally, exposing the first section of the implant 300 and the valve connectors. The physician then releases the advance button 142 and is able to view the location and operation of the implant 300 via fluoroscope, prior to releasing the implant 300. The implant 300 and delivery device 10 design provides the physician the ability see the implant 300 fully function prior to releasing the implant 300.

If the physician is unhappy with the operation of the implant 300, the physician depresses the retract button 144 and the implant 300 is retracted back into the delivery catheter 22 just as described during the loading sequence, except that loading tool 540 and adapter 560 are not used. If the physician is satisfied with the operation of the implant 300, the implant 300 is released from the connectors by first pulling a lock pin 541 and then operating the valve connector controls. The lock pin 541 prevents premature release of the implant by preventing the implanter from advancing the lead screw to a release point at which the implant is released.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A system comprising:
a cardiac implant;
a delivery device coupled to the cardiac implant comprising:
a control element having a distal end and a proximal end, the distal end attachable to the cardiac implant;
a delivery catheter surrounding the control element and having a distal end and a proximal end;
a handle assembly adjustably connected to the proximal end of the control element and to the delivery catheter, the handle assembly including:
a motor;
a power source capable of powering the motor; at least one control connected between the power source and the motor, usable to provide power having a first polarity that powers the motor in a first direction and usable to provide power having a second polarity that powers the motor in a second direction; and
a drive mechanism that moves the control element in a distal direction and the delivery catheter in a proximal direction relative to the handle assembly when the motor is powered in the first direction, and moves the control element in a proximal direction and the delivery catheter in the distal direction when the motor is powered in the second direction.

2. The system of claim 1, wherein the cardiac implant is a cardiac valve implant comprising:
a tubular braided support structure having an unfolded configuration and a folded configuration and including:
a proximal end formed of a plurality spires each having a high point and low point on either side of the high points;
a distal end;
first and second circumferential preformed folds between the distal and proximal ends of the tubular braided support structure, the circumferential preformed folds biasing the tubular braided support structure toward the folded configuration;
a valve leaflet assembly including:
a wireform including a plurality of commissural points;
valve material attached to the wireform;
wherein the wireform shapes the valve material into coapting valve leaflets when the valve material is attached to the wireform; and
wherein the valve leaflet assembly is attached to an inside surface of the support structure such that the commissural points of the wireform are aligned with the spires of the support structure.

3. The system of claim 2, wherein the low point between adjacent spires is shared by the adjacent spires and wherein when the support structure is in the folded configuration, the spires extend proximally of the second preformed fold.

4. The system of claim 2, wherein complete loops define distal ends of braids of the braided support structure and wherein the complete loops are connected to a braid that forms the high point of one of the plurality of spires.

5. The system of claim 2, wherein the wireform comprises one or more of:
the plurality of commissural points, the plurality of commissural points comprising tips having loops providing attachment points for the delivery device;
the wireform is nested within the support structure such that the valve material is located inside of the support structure; and
the wireform is formed of a single wire having ends connected together with a connector.

6. The system of claim 2, wherein each of the plurality of commissural points are separated by arcuate portions that include loops at a midpoint of the arcuate portions.

7. The system of claim 2, wherein the valve material comprises one or more of:
a plurality of sheets sewn together such that a proximal end of each sheet forms a leaflet; and
the valve material extends distally of the first preformed fold such that the valve material is folded with the first preformed fold.

8. The system of claim 2, further comprising a distal liner lining the inside surface of the support structure, distal of the second preformed fold.

9. The system of claim 2, wherein activating the motor in the first direction retracts the delivery catheter relative to the cardiac implant, thereby allowing the cardiac implant to expand within a target location.

10. A system comprising:
a cardiac implant;
a delivery system coupled to the cardiac implant for delivering the cardiac implant to a target site within a patient, the delivery system comprising:
a motorized delivery device having a handle, a delivery catheter assembly extending distally from the handle, and a motor contained within the handle and operable to pull a connector into a delivery catheter during a loading procedure and retract the delivery catheter relative to the cardiac implant during a delivery procedure;
a loading tray usable to assist in loading the cardiac implant into the delivery system, the loading tray comprising:
a handle compartment, the handle compartment sized and shaped to hold the handle in a desired position;
a basin usable to contain a solution during the loading procedure; and
a channel leading from the handle compartment to the basin, the channel sized to retain the delivery catheter during the loading procedure.

11. The system of claim 10, wherein the cardiac implant comprises a cardiac valve implant comprising:
a tubular braided support structure having an unfolded configuration and a folded configuration, the tubular braided support structure comprising:
first and second circumferential preformed folds between a distal end and a proximal end of the tubular braided support structure, the circumferential preformed folds biasing the tubular braided support structure toward the folded configuration;
a valve leaflet assembly including valve material attached to a wireform that shapes the valve material into coapting valve leaflets; and
wherein the valve leaflet assembly is attached to an inside surface of the support structure such that the valve material is protected from contact with the delivery catheter by the support structure.

12. The system of claim 11, wherein the tubular braided support structure proximal end includes a plurality of spires each having a high point and low point on either side of the high points.

13. The system of claim 12, wherein the plurality of spires comprises three spires.

14. The system of claim 13, wherein the valve leaflet assembly is attached to the inside surface of the support structure such that commissural points of the wireform are aligned with the spires of the support structure.

15. The system of claim 11, wherein complete loops define distal ends of braids of the braided support structure.

16. The system of claim 11, wherein the wireform comprises commissural points that include tips having loops providing attachment points for a delivery device.

17. A support structure for a cardiac valve implant comprising:
a proximal end formed of a plurality spires each having a high point and low point on either side of the high points;
a distal end;
first and second circumferential preformed folds between the distal and proximal ends of the support structure, the circumferential preformed folds biasing the support structure toward a folded configuration;
wherein when the support structure is in the folded configuration, the support structure forms a three-layered middle region, a single-layered proximal region, and a single-layered distal region; and
wherein the single-layered proximal region comprises proximal portions of a plurality of spires, which include the high points.

18. The support structure of claim 17, wherein the single-layered distal region comprises ventricular flare loops.

19. The support structure of claim 17, wherein the three-layered middle region extends from the first circumferential preformed fold to the second circumferential preformed fold and wherein the three-layered middle region comprises the low points.

20. The support structure of claim 17, wherein the single-layered distal region is one of an outer layer and an inner layer.
